(19) United States
(12) Patent Application Publication
(10) Pub. No.: US 2015/0050677 A1
(43) Pub. Date: Feb. 19, 2015

(54) METHODS AND SYSTEMS FOR MEASURING WHOLE BLOOD PLATELET REACTIVITY

(71) Applicant: University of Massachusetts Medical School, Boston, MA (US)

(72) Inventor: Jeffrey Rade, Worcester, MA (US)

(21) Appl. No.: 14/460,083

(22) Filed: Aug. 14, 2014

Related U.S. Application Data
(60) Provisional application No. 61/866,811, filed on Aug. 16, 2013.

(51) Int. Cl. G01N 33/86; G01N 33/72

(52) U.S. Cl. CPC G01N 33/86; G01N 33/721; G01N 2800/222; G01N 2800/226; G01N 2800/52
USPC 435/13

(57) ABSTRACT

The invention relates to a novel approach and related methods and systems, which provide accurate and easily implemented measurement and analyses of whole blood platelet reactivity, and impact on treatment thereof.

### PRIORITY CLAIMS AND RELATED PATENT APPLICATIONS

This application claims the benefit of priority from U.S. Provisional Application Ser. No. 61/866,811 filed on Aug. 16, 2013, the entire content of which is incorporated herein by reference in its entirety.

### TECHNICAL FIELD OF THE INVENTION

The invention generally relates to medical diagnosis and treatment. More particularly, the invention relates to a novel approach, and related methods and systems, which provide accurate and easily implemented measurement and analyses of whole blood platelet reactivity, and impact on treatment thereof.

### BACKGROUND OF THE INVENTION

Optimal inhibition of platelets is of vital importance in the prevention of myocardial ischemic complications in patients undergoing percutaneous coronary intervention. (Patti, et al. (2005) Circulation 111:2099-2106.) Dual antiplatelet therapy with aspirin plus a P2Y12 receptor inhibitor (e.g., clopidogrel, prasugrel) is essential to reducing ischemic complications and remains the standard of care after percutaneous coronary intervention.

Despite the use of dual antiplatelet therapy, a small but significant subgroup of patients still experience thrombotic events after stent placement. Such failures are often multifactorial in nature but closely correlate with the presence of clopidogrel high on-treatment platelet reactivity (HTPR). (Breet, et al. (2010) JAMA 303:754-62; Price, et al. (2008) Eur Heart J 29:992-1000.). The etiology of HTPR itself is multifactorial and influenced by baseline platelet reactivity, platelet turnover and genetic variability in clopidogrel bioactivation. As genetic predisposition only accounts for 12-25% of HTPR, functional platelet testing appears preferable to genetic screening for its identification. (Sweeny, et al. (2009) Nat Rev Cardiol 6:273-82.)

Substantial response variability has been reported with an incidence of poor clopidogrel response ranging from 5-44% depending on the population and definition. High on-treatment platelet reactivity (HTPR), despite clopidogrel therapy, is associated with a higher risk of adverse cardiovascular events including stent thrombosis. (Gurbel, et al. (2007) Thromb Res 120:311-21; Migliorini, et al. (2009) Circulation 120:2214-21.) The variability in clopidogrel response has prompted the clinical use of assays for platelet functional assessment to identify patients at increased risk of thrombotic complications and recurrent ischemic events. These high-risk patients may then be targeted with enhanced anti-platetelet therapy such as increased doses of clopidogrel or newer antiplatelet agents such as prasugrel or ticagrelor. (Bonello, et al. (2009) Am J Cardiol 103:5-10.)

Light transmission aggregometry (LTA) in platelet-rich plasma is considered the historical gold standard test to assess in vitro platelet activation and platelet-to-platelet aggregation. The sample is gently stirred and an agonist such as adenosine diphosphate (ADP), collagen, arachidonic acid, or epinephrine is added. Platelet aggregation leads to an increase in light transmission or sample impedance. These changes can be monitored over time and used to assess both primary and secondary platelet activation phase responses. Nonetheless, the technique is time consuming due to the requirement to produce platelet-rich plasma from whole blood, poorly standardized, requires highly trained personnel, repeated centrifugations that may traumatize platelets and a relatively large volume of blood sample.

The VerifyNow P2Y12 assay is a point-of-care test that is widely used to assess the efficacy of ADP-receptor antagonists and identify clopidogrel HTPR. (Price (2009) Circulation 119:2625-32.) The basis of the P2Y12 test is the ability of activated platelets to bind fibrinogen. Fibrinogen-coated microparticles aggregate in whole blood in proportion to the number of expressed platelet GP Ith/IIIa receptors. The rate of microbead aggregation is more rapid and reproducible if platelets are activated. Light transmittance increases as activated platelets bind and aggregate with fibrinogen-coated beads. The instrument measures ADP-induced platelet activation in citrated whole blood samples and reports results in P2Y12 reaction units (PRU).

Early versions of VerifyNow P2Y12 assay were also configured to simultaneously measure platelet reactivity to iso-thrombin receptor activating peptide as an indicator of maximal platelet activation not influenced by antiplatelet agents, and reported results in Base units (BASE). Studies have consistently demonstrated a correlation between the presence of clopidogrel HTPR as defined by elevated PRU over pre-defined thresholds and adverse outcome after PCI. (Brar, et al. (2011) J Am Coll Cardiol 58:1945-54.) These high-risk patients may theoretically be targeted for more aggressive anti-platelet therapy by either increasing the dose of clopidogrel or switching to newer ADP-receptor antagonists, such as prasugrel or ticagrelor, capable of more potent and reliable platelet inhibition. (Bonello, et al. (2009) Am J Cardiol 103:5-10.)

Unfortunately, such tailored approaches have thus far failed to improve clinical outcome, possibly due to the significant inter- and intra-individual variability of platelet reactivity assays. (Price, et al. (2011) JAMA 305:1097-105; Collet, et al. (2012) N Engl J Med 367:2100-9.)

There is an ongoing need for test that accurately identifies patients at increased risk of thrombotic events due to persistently elevated platelet reactivity despite the use of conventional therapy. More potent and expensive antiplatelet therapies could then be targeted to this patient group. It is strongly desired that novel approaches and techniques be developed that provide more accurate measurement of whole blood platelet reactivity.

### SUMMARY OF THE INVENTION

The invention provides a novel approach to improve the accuracy and hence, predictive value of whole blood platelet function tests. As the disclosure herein will demonstrate, current whole blood platelet reactivity assays do not provide accurate measurements of platelet reactivity due to confounding by a sample's hematocrit/hemoglobin level and cannot be successfully used to identify patients at high risk to whom newer antiplatelet therapies should be targeted.

The effect of hematocrit on whole blood aggregometry (e.g., VerifyNow PRU values) is demonstrated to be an in vitro phenomenon that is independent of intrinsic change in ADP-induced platelet reactivity and clopidogrel responsiveness. Correcting for hematocrit when using this assay may more accurately identify patients with HTPR that may benefit from alternative antiplatelet therapy.

ADP-induced platelet activation was measured using the VerifyNow P2Y12 assay, whole blood impedance and light transmission platelet aggregometry (LTA) before and after clopidogrel loading in 113 patients undergoing elective cardiac catheterization. Iso-TRAP-induced platelet activation was additionally measured using the VerifyNow device. Multivariate modeling employing clinical and laboratory variables was used to investigate the association between hematocrit and whole blood aggregometry (e.g. VerifyNow).

For examples, VerifyNow P2Y12 reaction units (PRU) and iso-TRAP Base units before and after clopidogrel loading, but not their relative change, exhibited strong negative correlation with hematocrit (p≦0.0005 for both). While hematocrit remained a strong predictor of post-clopidogrel PRU (p=0.001) in multivariate modeling, it was independent of post-clopidogrel ADP-induced platelet reactivity as measured by LTA (p=0.001). Correcting for the effects of hematocrit resulted in a 15-39% reduction in the prevalence of HTPR defined by thresholds of 208-236 PRU.

In one aspect, the invention generally relates to a method for measuring a subject's whole blood platelet reactivity. The method includes: providing a first sample of fresh whole blood of the subject; applying an ADP agonist to the sample to induce platelet activation; measuring an apparent level of platelet reactivity induced by the ADP agonist; providing a second sample of fresh whole blood of the subject; measuring a level of hematocrit and/or hemoglobin of the second sample; and transforming the apparent level of platelet reactivity by the level of hematocrit and/or hemoglobin to obtain the actual platelet reactivity of the subject.

In another aspect, the invention generally relates to a method for assessing thrombosis risk of a subject in response to clopidogrel therapy. The method includes: providing a first sample of fresh whole blood of the subject; applying an ADP agonist to the sample to induce platelet activation; measuring an apparent level of platelet reactivity induced by the ADP agonist; providing a second sample of fresh whole blood of the subject; measuring a level of hematocrit and/or hemoglobin of the second sample; and transforming the apparent level of platelet reactivity by the level of hematocrit and/or hemoglobin to assess thrombosis risk of a subject.

In yet another aspect, the invention generally relates to a method for assessing thrombosis risk of a subject in response to ticagrelor therapy. The method includes: providing a first sample of fresh whole blood of the subject; applying an ADP agonist to the sample to induce platelet activation; measuring an apparent level of platelet reactivity induced by the ADP agonist; providing a second sample of fresh whole blood of the subject; measuring a level of hematocrit and/or hemoglobin of the second sample; and transforming the apparent level of platelet reactivity by the level of hematocrit and/or hemoglobin to assess thrombosis risk of a subject.

### BRIEF DESCRIPTION OF THE DRAWINGS

FIG. 1 shows the effect of hematocrit on VerifyNow P2Y12 assay results before (Pre) and after (Post) clopidogrel loading. Correlation between hematocrit and: A) Base units (BASE), R₂=0.38, p<0.0001 and R₂=0.44, p<0.0001 for Pre (n=108) and Post (n=98) values, respectively; B) P2Y12 reaction units (PRU), R₂=0.46, p<0.0001 and R₂=0.12, p=0.0005 for Pre- (n=108) and Post- (n=98) values, respectively, and; C) Change in PRU (n=93, p=0.41). 95% confidence intervals are shown for all correlations.

FIG. 2 shows the effect of hematocrit on ADP-induced platelet activation before (Pre) and after (Post) clopidogrel loading. Correlation between hematocrit and: A) Aggregation in RESPONSE TO 10 μM ADP MEASURED BY WHOLE BLOOD AGGREGOMETRY (WBA), R₂=0.17, P<0.0001 and R₂=0.08, p<0.003 for Pre (n=115) and Post (n=113) values, respectively); B) Aggregation in response to 20 μM ADP measured by light transmission aggregometry (LTA), R₂=0.0006, p=0.8 and R₂=0.04, p=0.03 for pre (n=114) and post (n=113) values, respectively); C) Residuals of the regression between WBA and LTA values (n=93, p<0.0001), and; D) Change in aggregation with clopidogrel loading measured by WBA (n=107, p=0.07) and LTA (n=105, p=0.62), (n=93, p=0.41). 95% confidence intervals are shown for all correlations.

### DETAILED DESCRIPTION OF THE INVENTION

The invention provides a novel approach for accurate and easily implemented measurement and analyses of whole blood platelet reactivity, and impact on treatment thereof. The invention enables increased clinical ability of whole blood platelet reactivity assays in detecting patients at risk of thrombotic events who may benefit from enhanced antiplatelet therapy. The invention improves the currently limited clinical utility of platelet function assays performed in whole blood samples by applying a correction algorithm based on the prevelant levels of hematocrit/hemoglobin. The present invention is expected to increase the assay's ability to identify patients who are truly clinically at risk of thrombotic events and who may therefore benefit from enhanced medical therapy. Furthermore, patients with adequately suppressed platelets on conventional therapy will be more accurately identified, with reduced risk of misclassification that would lead to unnecessary exposure to the bleeding risk and cost of enhanced antipaltelet therapies.

An early validation study suggested that the VerifyNow P2Y12 assay is unaffected by most patient factors, including hematocrit. (Malinin, et al. (2006) Methods Find Exp Clin Pharmacol 28:315-22.) Subsequent studies, however, revealed that age, female gender, diabetes mellitus and hematocrit are among the variables associated with elevated PRU values. (Elsenberg, et al. (2009) Thromb Haemost 102:719-27; Voisin, et al. (2011) Thromb Haemost 106:227-9; Price, et al. (2009) Am J Cardiol 103:1339-43.; Tidjane, et al. (2011) J Thromb Haemost 9:614-6.)

Unlike prior studies, the present study analyzed the relationship of clinical and laboratory factors to PRU as a continuous variable, rather than dichotomously based on the presence or absence of HTPR defined by a particular PRU threshold. Multivariate modeling in our study population found age to correlate with pre- but not with post-clopidogrel PRU values. While female gender and diabetes also correlated with PRU, both were found to be covariates of hematocrit.

A recent study examining the effect of anemia on clopidogrel responsiveness found no apparent association between hemoglobin and PRU values at baseline, though higher values were observed in anemic patients after clopidogrel loading. (Toma, et al. (2012) Am J Cardiol 109:1148-53.) A lower absolute reduction in platelet reactivity was also observed in anemic versus non-anemic subjects following clopidogrel loading, as assessed by both LTA and the VerifyNow assay.

With the benefit of a larger cohort of subjects with serial pre- and post-procedure complete blood counts and analysis of hematocrit as a continuous rather than dichotomous variable, a significant effect of hematocrit on PRU and BASE values both before and after clopidogrel loading was observed in the present study. In contrast to the prior study, no correlation was found between baseline hematocrit and change in PRU with clopidogrel loading and no difference between the absolute decrease in PRU values between anemic patients and those with normal hemoglobin.

A critical question is whether the observed correlation between hematocrit and VerifyNow PRU is due to a hematocrit-dependent biological change in intrinsic platelet reactivity or to an in vitro phenomenon inherent to the assay. The examples disclosed herein provide strong evidence for the latter.

First, the effect of hematocrit on apparent ADP-induced platelet reactivity is more pronounced with whole-blood assays such as the VerifyNow and WBA than it is for LTA, which is performed in the absence of red blood cells.

Second, the small effect of hematocrit on ADP-induced platelet aggregation measured by LTA that have been observed is also likely to be artifactual. (Toma, et al. (2012) Am J Cardiol 109:1148-53; Cecchi, et al. (2009) Am J Cardiol 104:764-8.) Platelet rich plasma is generated from blood mixed with a fixed ratio of citrate. Because citrate is excluded from red blood cells, the effective plasma citrate concentration and therefore the free calcium concentration that supports platelet aggregation, varies with hematocrit. (Mollison, et al. (1958) Lancet 1:766-9.) Kelton et al. showed that the apparent association between hematocrit and ADP-induced platelet activation assessed by LTA is eliminated by normalizing the plasma citrate concentration, which is rarely done when performing platelet aggregometry. (Kelton, et al. (1980) Blood 56:38-41.)

Third, hematocrit had no effect on the magnitude of change in ADP-induced platelet aggregation after clopidogrel loading measured by VerifyNow PRU, WBA or LTA.

Additionally, the VerifyNow assay depends on changes in whole blood turbidity as ADP-activated platelets adhere to human fibrinogen-coated beads. The turbidity of a suspension is the overall result of absorption and scattering effects that are dependent on the size and distribution of solid particles in the suspension. (Whitlock (1947) Blood 2:463-73.) The algorithm to determine platelet reactivity based on changes in turbidity likely depends on assumptions including the nature and number of light-interfering red blood cells in the sample such that deviations in the sample hematocrit from the established standard may alter the algorithm's accuracy. This may also explain why platelet and white cell count remained significant predictors of VerifyNow PRUs in clopidogrel naive patients.

Furthermore, most compelling of all, hematocrit was an independent predictor of post-clopidogrel PRU in multivariate modeling (Table 3, Model 1), even when LTA was included in the model to account for the clopidogrel-induced changes in intrinsic platelet reactivity (Model 2).

The finding that the association between hematocrit and VerifyNow PRU appeared largely due to be an in vitro effect of the assay provided the rationale for devising a system (e.g., a procedure including a designed algorithm) to correct for the effects of hematocrit. In an exemplary system, subtracting 7.5 PRU for every hematocrit percentage point below and adding 7.5 PRU for every percentage point above 42%, abolished the effect of hematocrit on both the VerifyNow BASE and PRU results and resulted in the reclassification of a substantial number of subjects with respect to the presence or absence of HTPR. While clopidogrel HTPR defined by the VerifyNow P2Y12 assay has been convincingly demonstrated to be associated with adverse outcomes at a population level, modifying individual therapy based on assay results has failed to translate into improved patient outcomes. (Brar, et al. (2011) J Am Coll Cardiol 58:1945-54; Price, et al. (2011) JAMA 305:1097-105; Collet, et al. (2012) N Engl J Med 367:2100-9; Trenk, et al. (2012) J Am Coll Cardiol 59:2159-64.) Correcting for the effects of low hematocrit, which is frequently common after PCI, may improve the accuracy of identifying patients with clopidogrel HTPR who could benefit from more potent antiplatelet therapy and avoid aggressive treatment of patients who do not.

Examples from two recent clinical trials illustrate the potential importance of accounting for the effects of hematocrit when assessing HTPR. The TRIGGER-PCI trial used the VerifyNow P2Y12 assay to identify patients undergoing PCI with clopidogrel HTPR and randomize them to either continuation of clopidogrel or more intense antiplatelet therapy with prasugrel. (Trenk, et al. (2012) J Am Coll Cardiol 59:2159-64.) Of the 3283 patients screened, 625 were classified as having HTPR based on a PRU value >208 and eligible for randomization. A similar 8% classification error rate caused by hematocrit that was observed in our study cohort would have been expected to reclassify 262 TRIGGER-PCI subjects, or 42% of those that were eligible for randomization. The trial was stopped prematurely because no difference in 6-month outcomes was observed between the two antiplatelet regimens.

The ARTIC trial randomized 2440 PCI patients to either assay-guided or conventional antiplatelet therapy. (Collet, et al. (2012) N Engl J Med 367:2100-9.) Of the 1213 patients in the assay-guided group, 419 (35%) had clopidogrel HTPR defined by a PRU≧235. At that PRU threshold, 133 (11%) of screened subjects, representing 32% of those targeted for additional antiplatelet therapy, would have erroneously been identified as having HTPR. The ARTIC trial found no improvement in outcome with assay-guided compared to conventional therapy. It is not inconceivable that the results of both trials could have been confounded by inaccuracies in identifying subjects with clopidogrel HTPR using the VerifyNow P2Y12 assay.

In one aspect, the invention generally relates to a method for measuring a subject's whole blood platelet reactivity. The method includes: providing a first sample of fresh whole blood of the subject; applying an ADP agonist to the sample to induce platelet activation; measuring an apparent level of platelet reactivity induced by the ADP agonist; providing a second sample of fresh whole blood of the subject; measuring a level of hematocrit and/or hemoglobin of the second sample; and transforming the apparent level of platelet reactivity by the level of hematocrit and/or hemoglobin to obtain the actual platelet reactivity of the subject.

In certain embodiments, the first sample of fresh whole blood and the second sample of fresh whole blood are obtained from the subject at the same time.

In certain embodiments, the first sample of fresh whole blood and the second sample of fresh whole blood are obtained from the subject at different times less than 48 hours part (e.g., less than 36 hours apart, less than 24 hours apart, less than 12 hours apart).

ADP receptor inhibitor is a class of antiplatelet agents, which inhibit some or all types of adenosine diphosphate receptors (P2Y receptors). P2Y receptors are a family of purinergic G protein-coupled receptors, stimulated by nucleotides such as ATP, ADP, UTP, UDP and UDP-glucose. Inhibitors of the receptor subtype P2Y12 are one class of P2Y receptor inhibitors. These drugs include clopidogrel, prasugrel, ticlopidine, ticagrelor, cangrelor, and elinogrel.

In certain embodiments, measuring an apparent level of platelet reactivity impaired by the ADP agonist generates a PRU value. In certain embodiments, wherein transforming the apparent level of platelet reactivity by the level of hematocrit and/or hemoglobin to obtain the actual platelet reactivity of the subject comprises applying a correction algorithm to normalize a PRU value by the level of hematocrit and/or hemoglobin. In certain embodiments, normalizing a PRU value comprises subtracting a fixed value of PRU for every hematocrit percentage point below and adding the fixed value of PRU for every percentage point above (e.g., 10%, 15%, 20%, 25%, 30%, 40%, 42%, 45%, 50%).

In another aspect, the invention generally relates to a method for assessing thrombosis risk of a subject in response to clopidogrel therapy. The method includes: providing a first sample of fresh whole blood of the subject; applying an ADP agonist to the sample to induce platelet activation; measuring an apparent level of platelet reactivity induced by the ADP agonist; providing a second sample of fresh whole blood of the subject; measuring a level of hematocrit and/or hemoglobin of the second sample; and transforming the apparent level of platelet reactivity by the level of hematocrit and/or hemoglobin to assess thrombosis risk of a subject.

In yet another aspect, the invention generally relates to a method for assessing thrombosis risk of a subject in response to ticagrelor therapy. The method includes: providing a first sample of fresh whole blood of the subject; applying an ADP agonist to the sample to induce platelet activation; measuring an apparent level of platelet reactivity induced by the ADP agonist; providing a second sample of fresh whole blood of the subject; measuring a level of hematocrit and/or hemoglobin of the second sample; and transforming the apparent level of platelet reactivity by the level of hematocrit and/or hemoglobin to assess thrombosis risk of a subject.

### Examples

The experimental results establish a strong effect of hematocrit on VerifyNow P2Y12 assay results both prior to and following clopidogrel loading. Evidence indicates that this effect is predominantly an in vitro phenomenon that is independent of intrinsic changes to platelet reactivity. Correcting for the effects of hematocrit may improve the accuracy of the VerifyNow P2Y12 assay at identifying patients with HTPR and guiding modifications to their antiplatelet therapy.

In yet another aspect, the invention also relates to a system that concurrently measures whole blood sample platelet reactivity and hematocrit/hemoglobin and then uses the results of the hematocrit/hemoglobin to modify the results of the whole blood sample platelet reactivity.

### Materials and Methods

### Subjects

Certain data discussed herein were obtained from patients undergoing elective cardiac catheterization at the Johns Hopkins Hospital between August, 2008 and December, 2009. (Linnemann, et al. (2010) Ann Hematol 89:597-605.)

In addition to the platelet function studies proscribed in the multicenter protocol, patients enrolled at Johns Hopkins Hospital underwent assessment of clopidogrel responsiveness measured by the VerifyNow P2Y12 assay (Accumetrics, San Diego, USA) as well as measurement of plasma fibrinogen and vonWillibrand factor. The study was approved by the Johns Hopkins human subjects review board and all subjects provided written consent. Subjects were eligible for the Design Verification Study if they were ≧18 years of age, had established cardiovascular disease or at least two of eight risk factors (current or prior tobacco smoking, hypertension, hyperlipidemia, family history of premature vascular disease, post-menopausal female, diabetes mellitus, morbid obesity or sedentary lifestyle). Exclusion criteria included: inability to provide written informed consent, treatment with any platelet inhibiting drug other than aspirin, inherited or acquired platelet function disorder, vonWillebrand disease, or current participation in another trial with a drug known to affect platelet function or any investigational drug.

After collection of baseline blood samples but prior to coronary angiography, subjects were administered a loading dose of 300-600 mg clopidogrel. Percutaneous coronary intervention (PCI) was performed at the same time on an ad hoc basis at the discretion of the treating physician. Patients were excluded from further analysis if a glycoprotein IIb/IIIa antagonist was used during PCI.

### Blood Collection and Analyses

Blood samples were collected at baseline, prior to clopidogrel administration, and 6-24 hours later, after the catheterization procedure. After discarding the first 10 ml, blood was collected into siliconized glass vacutainers containing 3.2% sodium citrate (for platelet function testing and measurement of fibrinogen and vonWillebrand factor antigen) or EDTA-containing vacutainers (for complete blood counts). The samples were maintained at room temperature and hand-delivered to the laboratory where platelet function analyses were performed within 60 minutes of collection. Aliquots of centrifuged platelet-poor plasma and serum were stored at −70° C. until batch analyzed.

Citrated blood was centrifuged to obtain platelet-rich plasma (PRP) and adjusted to a final platelet count of 180,000 mm⁻³ as previously described. (Nazarian, et al. (2009) Thromb Res 126:379-83.) Light transmission aggregometry (LTA) was performed on PRP stimulated with 20 μM adenosine diphosphate (ADP) using a Chrono-Log Model 560CA aggregometer (Chrono-Log, Havertown, Pa.). Whole blood impedance aggregometry (WBA) was performed on citrated blood stimulated with 1004 ADP also using a Chrono-Log aggregometer. Complete blood counts were measured with an XE-2100 automated analyzer (Sysmex Corporation, Kobe, Japan). Anemia was defined as hemoglobin concentrations <11.7 g/dL for women and <12.9 g/dL as previously reported. (Toma, et al. (2012) Am J Cardiol 109:1148-53.)

The VerifyNow P2Y12 assay was performed on citrated blood according to the manufacturer's instructions. Clopidogrel responsiveness was assessed by ADP-induced platelet aggregation and recorded as PRU, whereas maximal platelet reactivity was assessed by stimulation with iso-thrombin receptor activating peptide agonist and recorded as Base units (BASE). Plasma vonWillebrand factor (vWF) antigen was assessed by an immunoturbometric assay using the STA®-Liatest®VWF:Ag kit (Diagnostica Stago, Asnieres, France) and expressed as percent of normal control plasma. Plasma fibrinogen was measured by a modified Clauss method using the Multifibren U kit (Siemens Healthcare Diagnostics Products GmbH, Marburg, Germany) and expressed in mg/dl. Both coagulation tests were performed on a Siemens BCS Coagulation Analyzer.

### Statistical Analysis

Continuous data are shown as mean±standard deviation or median±interquartile range and categorical variables are presented as percentages. Data and regression residuals were tested for normality using the Shapiro-Wilk test. The paired two-sample t-test was used to compare normal pre- and post-continuous data. The Wilcoxon signed-rank test was used for skewed data. Tukey's ladder of powers was used to guide transformations of skewed data. Data that remained skewed were treated dichotomously or categorically, using reference ranges established by the Johns Hopkins Medical Laboratory to set category limits. Data transformation was avoided if regression to the predictor yielded normally distributed residuals.

Univariate linear regression for VerifyNow pre- and post-clopidogrel results was performed using laboratory and clinical candidate predictors. Variables with p≦0.2 were included in the initial multivariate models. The Furnival-Wilson leaps-and-bounds algorithm was used for subset variable selection and the model optimized based on Akaike's Information Criteria (AIC). The Tobit model was used in cases of censoring in the dependent variable.

All statistical analyses were performed using Stata/SE 12 for Windows (StataCorp, College Station, Tex., USA). Unless otherwise specified, all tests were 2-sided with significance set at α=0.05.

### Study Population Characteristics

A total of 156 subjects undergoing cardiac catheterization met the inclusion/exclusion criteria of the parent study and had baseline blood samples obtained prior to clopidogrel loading. Thirty-seven subjects were subsequently excluded due to receiving a glycoprotein IIb/IIIa antagonist during PCI (n=21), technical difficulties obtaining/processing post-clopidogrel loading blood samples (n=15) or refusal of continued participation (n=1). An additional six subjects did not have valid VerifyNow P2Y12 results for technical reasons. Consequently, 113 subjects with pre- and post-clopidogrel blood samples formed the study cohort for this present analysis. The baseline characteristics of these subjects are summarized in Table 1.

TABLE 1
Baseline characteristics of the 113 study subjects

| | Number of | Mean ± SD/ |
|---|---|---|
| Characteristic | patients (%) | median (IQR) |
| Age (years) | | 62.6 ± 10.5 |
| Male gender | 81 (72%) | |
| Body Mass Index (kg/m²) | | 30.1 (25.3-34.3) |
| Race | | |
| White | 99 (88%) | |
| African American | 8 (7%) | |
| Other | 7 (5%) | |
| Diabetes mellitus | 30 (27%) | |
| Hypertension | 74 (66%) | |
| Hyperlipidemia | 78 (69%) | |
| Family history of CAD | 57 (50%) | |
| Post-menopausal women | 26 (81%) | |
| Smoking status | | |
| Non-smoker | 63 (56%) | |
| Current smoker | 33 (29%) | |
| Ex-smoker | 17 (15%) | |
| Known vascular disease | | |
| Coronary artery disease | 101 (89%) | |
| Cerebrovascular disease | 2 (2%) | |
| Peripheral vascular disease | 7 (6%) | |
| Aspirin therapy | 100 (89%) | |
| 81 mg | 75 | |
| 162 mg | 1 | |
| 325 mg | 24 | |
| Anemia* | 21 (18.6%) | |
| Baseline hemoglobin (g/dl) | | 13.9 ± 1.6 |
| Baseline hematocrit (%) | | 41.0 ± 4.3 |
| | | (range 27.7 to 52.4) |
| White blood cell count (10⁶/ml) | | 7.6 (5.6-8.6) |
| Platelets (10⁶/mm³) | | 228 (179-258) |

*Hemoglobin concentrations < 11.7 g/dL for women and < 12.9 g/dl. (Toma, et al. (2012) Am J Cardiol 109:1148-53.)
CAD = coronary artery disease;
IQR = Interquartile range;
SD = standard deviation

Two subjects were loaded with 300 mg of clopidogrel while the remaining 111 were loaded with 600 mg. Fifty-four subjects (48%) subsequently underwent PCI during the index procedure. Compared to pre-clopidogrel values, the mean VerifyNow PRU was significantly lower (287±56 versus 179±90, p<0.0001) whereas the mean BASE was significantly higher (279±47 versus 298±53, p<0.0001) following clopidogrel loading. There was also a small but significant drop in mean hematocrit observed after the catheterization procedure (41.0±4.3% to 38.9±4.5%, p<0.0001).

### Effect of Hematocrit and Other Variables on VerifyNow Results

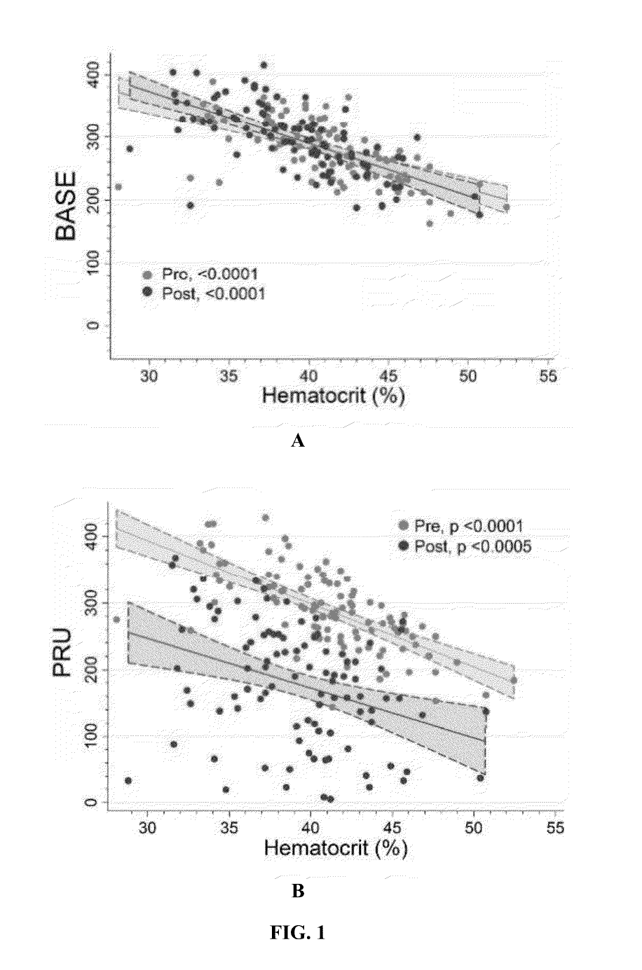

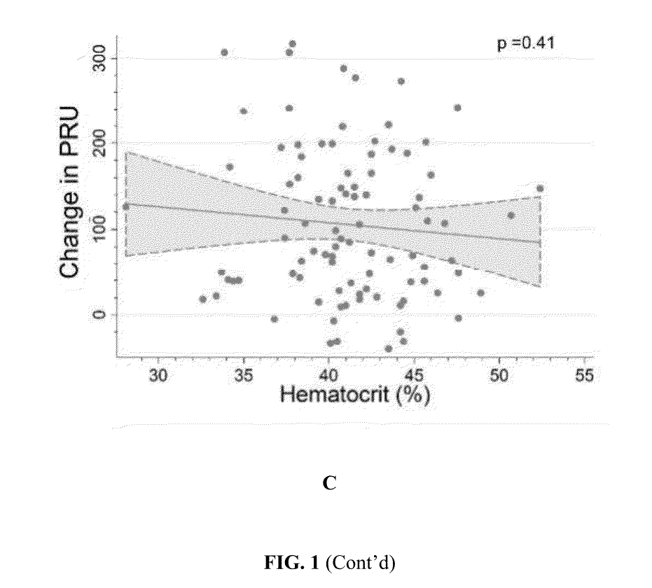

Strong negative correlations were observed between hematocrit and both BASE and PRU results at baseline, which persisted after clopidogrel loading (FIGS. 1A and B). Univariate analyses identified hematocrit, age, gender, platelet count and hypertension as correlating with PRU prior to clopidogrel loading whereas hematocrit was the only factor to correlate with PRU following clopidogrel loading (Table 2). In contrast to a previous report, the change in PRU following clopidogrel loading was independent of baseline hematocrit (FIG. 1C and Table 2) with no difference between subjects with and without anemia (mean PRU 106±89 vs. 107±85, respectively, p=0.96). (Toma, et al. (2012) Am J Cardiol 109:1148-53.)

TABLE 2
Univariate regression analysis of variables potentially associated with VerifyNow PRU results before
and after clopidogrel loading. Coefficients are shown for statistically significant results

| | PRU Pre-clopidogrel | | PRU Post-clopidogrel | | Change in PRU | |
|---|---|---|---|---|---|---|
| Variable | Coefficient | p-value | Coefficient | p-value | Coefficient | p-value |
| Age (years) | 1.77 | 0.0006 | | 0.34 | | 0.18 |
| Male gender | −52.6 | <0.0001 | | 0.17 | | 0.43 |
| Race (white vs. other) | | 0.44 | | 0.89 | | 0.67 |
| Aspirin dose (81 mg vs. | | 0.96 | | 0.28 | | 0.73 |
| higher) | | | | | | |
| WBC (10³/mm³) | | 0.07 | | 0.92 | | 0.52 |
| Hematocrit (%) | −9.5 | <0.0001 | −7.5 | 0.0005 | | 0.41 |
| MCV (<90 vs. ≧90 fL) | | 0.08 | | 0.18 | | 0.67 |
| RDW (<14.5 vs. ≧14.5%) | | 0.23 | | 0.74 | | 0.77 |
| Platelet count (10³/mm³) | 0.30 | 0.0004 | | 0.10 | | 0.49 |
| MPV (fL) | | 0.70 | | 0.73 | | 0.84 |
| Fibrinogen (log mg/dL)* | | 0.96 | | 0.22 | | 0.16 |
| vWF antigen (log %)* | | 0.29 | | 0.32 | | 0.73 |
| Hypertension | 38.13 | 0.001 | | 0.05 | | 0.97 |
| Hyperlipidemia | | 0.76 | | 0.62 | | 0.31 |
| Diabetes mellitus | | 0.13 | 47.6 | 0.02 | | 0.17 |
| PCI during index | | | | 0.38 | | 0.29 |
| procedure | | | | | | |
| LTA (%) | | 0.41 | 2.0 | <0.0001 | | |
| Change in LTA (%) | | | | | 1.1 | <0.001 |

*n = 64.
CVA = cerebrovascular accident (stroke); LTA = light transmission aggregometry; MCV = mean cell volume; MPV = mean platelet volume; PCI = percutaneous coronary intervention; WBC = white blood cell count.

To further define the relationship between hematocrit and VerifyNow results, multivariate modeling was performed (Table 3). Hematocrit, age, white cell count and platelet count were independently associated with pre-clopidogrel PRU whereas hematocrit was the only variable to correlate with PRU after clopidogrel loading (Model 1). Hematocrit remained independently associated with post-clopidogrel PRU even after ADP-induced platelet aggregation, as measured by LTA, was included in the model to account for the effect of clopidogrel on intrinsic platelet reactivity (Model 2). Gender and diabetes also exhibited strong covariate interactions with hematocrit (post-procedure hematocrit 35.5±3.5% in females vs. 40.2±4.2% in males, p<0.0001 and 37.0±5.1 in diabetics vs. 39.6±4.0 in non-diabetics, p=0.006) but neither were independently associated with either pre- or post-clopidogrel PRU.

TABLE 3
Multivariate regression analysis of factors potentially associated
with VerifyNow PRU before and after clopidogrel loading

| | PRU Pre- | | PRU Post- | | PRU Post- | |
|---|---|---|---|---|---|---|
| | clopidogrel* | | clopidogrel Model 1^{†} | | clopidogrel Model 2^{‡} | |
| Variable | Coefficient | p-value | Coefficient | p-value | Coefficient | p-value |
| Age, years | 1.1 | 0.006 | | | | |
| Gender, male | | NI | | NI | | NI |
| WBC (10³/mm³) | −2.55 | 0.002 | | | | |
| Hematocrit (%) | −7.95 | <0.001 | −7.2 | 0.001 | −6.2 | <0.001 |
| MCV (<90 vs. ≧90 fL) | | NI | | NI | | NI |
| Platelet count | 0.30 | <0.001 | | NI | | NI |
| (10³/mm³) | | | | | | |
| Hypertension | | 0.09 | | 0.06 | | NI |
| Diabetes mellitus | | NI | | NI | | NI |
| LTA (%) | | | | | 1.8 | <0.001 |

*p < 0.0001, R² = 0.63;
^{†}p = 0.0004, R₂ = 0.16;
^{‡}p <0.0001, R² = 0.37.
WBC = white blood cells; MCV = mean cell volume; LTA = light transmission aggregometry; MPV = mean platelet volume; NI = not included in optimized model.

### Differential Effect of Hematocrit on ADP-Induced Platelet Aggregometry

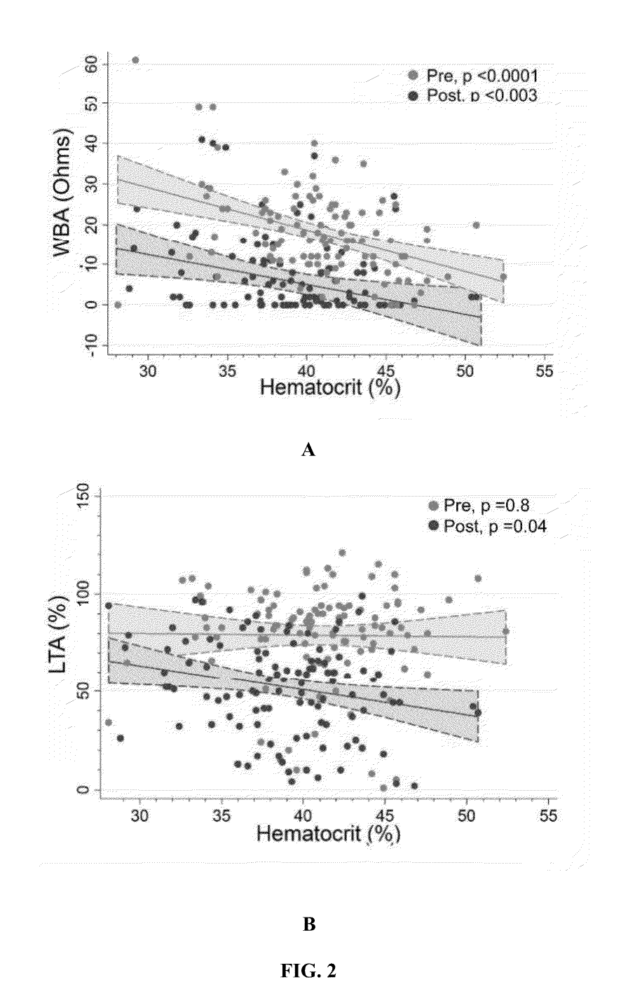

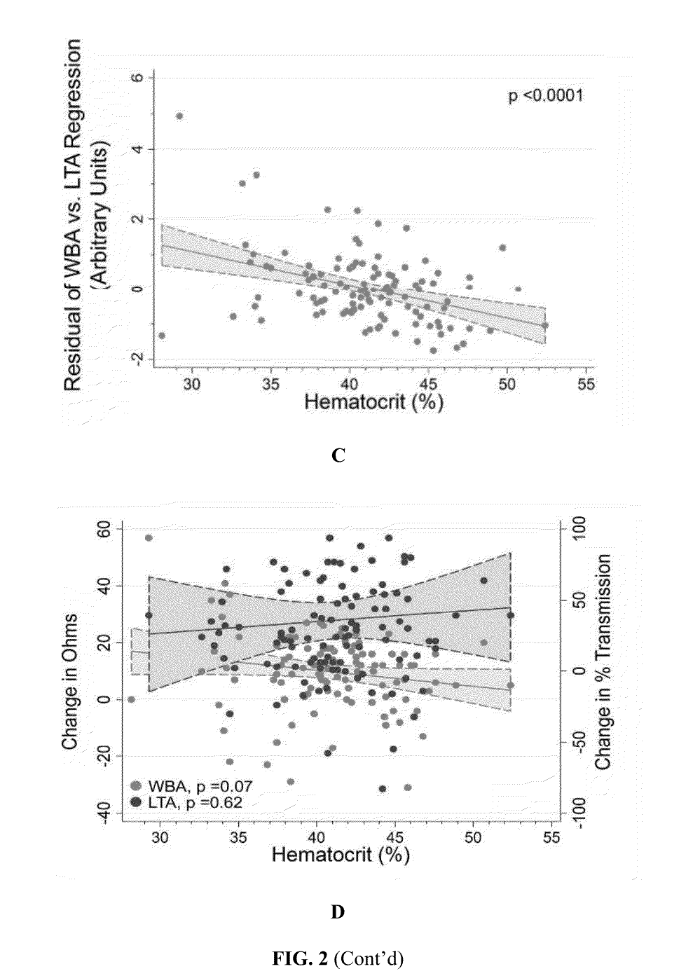

Clopidogrel loading resulted in a significant reduction in ADP-induced platelet aggregation: By whole blood impedance aggregometry (WBA) median aggregation to 10 μM ADP fell from 16 (IQR;11,24) to 4 (IQR; 0,12) ohms (P<0.0001) while by light transmission aggregometry (LTA), median aggregation in response to 20 μM ADP fell from 84 (IQR;71, 94) to 52 (IQR;37,70) percent (P<0.0001). Similar to the VerifyNow P2Y12 assay, there was a strong negative correlation between hematocrit and ADP-induced platelet reactivity assessed by WBA, both before and after clopidogrel loading (FIG. 2A). In contrast, the effect of hematocrit on ADP-induced platelet reactivity assessed by LTA was much less pronounced, with no significant effect noted prior to clopidogrel loading and only a relatively small effect observed afterwards (FIG. 2B). This disproportionate effect of hematocrit on ADP-induced platelet activation measured in whole blood compared to platelet rich plasma systematically affected the linear relationship between pre-clopidogrel WBA and LTA values (FIG. 2C). Hematocrit did not affect the change in ADP-induced platelet activation following clopidogrel loading measured by either WBA or LTA (FIG. 2D), similar to that observed when measured by the VerifyNow P2Y12 assay. In aggregate, these data show that hematocrit does not intrinsically alter ADP-induced platelet reactivity or its inhibition by clopidogrel to any appreciable degree.

### Correction of PRU Values for Hematocrit and Effect on Prevalence of HTPR

Given that the association between hematocrit and PRU values appears largely to be an in vitro effect of the VerifyNow assay, the utility of using a correction algorithm to normalize PRU values for the influence of hematocrit was investigated. A correction coefficient, 7.5 PRU/percent decrease in hematocrit, was derived from the linear regression of aggregate pre and post-clopidogrel BASE-hematocrit pairs and used to transform post-clopidogrel PRU values around the hematocrit pivot point of 42%, the lower limit of normal hematocrit in our laboratory. As the mean hematocrit post-PCI was 38.9±4.5%, the predominant effect of this correction was to lower PRU values in nearly three quarters of subjects and to raise it in most others. Correction also resulted in the reclassification of 8-13% of all subjects with respect to the presence or absence of HTPR using the range of PRU thresholds (178-236) that have been employed in several recent clinical studies (Table 4). (Breet, et al. (2010) JAMA 303:754-62; Price, et al. (2011) JAMA 305:1097-105; Collet, et al. (2012) N Engl J Med 367:2100-9; Price, et al. (2011) Circulation 124:1132-7; Kelton, et al. (1980) Blood 56:38-41; Trenk, et al. (2012) J Am Coll Cardiol 59:2159-64.) HTPR reclassification rates (9-13%) were similar among subjects who underwent PCI during the index procedure. At thresholds of 208-236 PRU that are commonly used in clinical practice to define clopidogrel responsiveness, correction for hematocrit reduced the prevalence of HTPR in the present study cohort by 15-39% (Table 3).

TABLE 4
Number of subjects with clopidogrel HTPR before and after correction for
hematocrit using published PRU thresholds

| PRU Threshold for | HTPR by original | HTPR by Corrected | |
|---|---|---|---|
| HTPR | PRU | PRU | Reclassified |
| ≧178 [c] | 50 (51%) | 46 (48%) | 8 (8%) |
| ≧208 [d] | 39 (40%) | 33 (34%) | 8 (8%) |
| ≧230 [e] | 31 (32%) | 21 (22%) | 10 (10%) |
| ≧235 [b] | 30 (31%) | 19 (20%) | 11 (11%) |
| ≧236 [a] | 30 (31%) | 18 (19%) | 12 (13%) |

HTPR = high clopidogrel on-treatment platelet reactivity; PRU = P2Y12 reaction unit.
[a] - Breet, et al. (2010) JAMA 303:754-62.
[b] - Collet, et al. (2012) N Engl J Med 367:2100-9.
[c] - Gurbel, et al. (2012) JAMA 308:1785-94.
[d] - Price, et al. (2011) Circulation 124:1132-7; Trenk, et al. (2012) J Am Coll Cardiol 59:2159-64.
[e] - Brar, et al. (2011) J Am Coll Cardiol 58:1945-54; Price, et al. (2011) JAMA 305:1097-105.

The major findings include: (1) VerifyNow PRU and BASE results strongly correlated with hematocrit, both before and after clopidogrel loading; (2) In contrast to prior reports, hematocrit did not influence clopidogrel responsiveness as defined by change in PRU with clopidogrel loading; (3) Hematocrit disproportionately affected ADP-induced platelet aggregation when assessed in whole blood compared to platelet-rich plasma; (4) Hematocrit retained additive predictive value for VerifyNow PRU beyond ADP-induced platelet reactivity in multivariate analysis, and; (5) Correcting for hematocrit resulted in reclassification of a substantial percentage of subjects with respect to the presence or absence of HTPR and reduced its overall prevalence as defined by accepted PRU thresholds.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A method for measuring a subject's whole blood platelet reactivity, comprising:
providing a first sample of fresh whole blood of the subject;
applying an adenosine diphosphate agonist to the sample to induce platelet activation;
measuring an apparent level of platelet reactivity induced by the ADP agonist;
providing a second sample of fresh whole blood of the subject;
measuring a level of hematocrit and/or hemoglobin of the second sample; and
transforming the apparent level of platelet reactivity by the level of hematocrit and/or hemoglobin to obtain the actual platelet reactivity of the subject.

2. The method of claim 1, wherein the first sample of fresh whole blood and the second sample of fresh whole blood are obtained from the subject at the same time.

3. The method of claim 1, wherein the first sample of fresh whole blood and the second sample of fresh whole blood are obtained from the subject at different times less than 48 hours part.

4. The method of claim 1, wherein measuring an apparent level of platelet reactivity induced by the adenosine diphosphate agonist generates a PRU value.

5. The method of claim 5, wherein transforming the apparent level of platelet reactivity by the level of hematocrit and/or hemoglobin to obtain the actual platelet reactivity of the subject comprises applying a correction algorithm to normalize a PRU value by the level of hematocrit and/or hemoglobin.

6. The method of claim 6, wherein normalizing a PRU value comprises subtracting a fixed value of PRU for every hematocrit percentage point below and adding the fixed value of PRU for every percentage point above 40%.

7. A method for assessing thrombosis risk of a subject in response to clopidogrel therapy, comprising:
providing a first sample of fresh whole blood of the subject;
applying an adenosine diphosphate agonist to the sample to induce platelet activation;
measuring an apparent level of platelet reactivity induced by the ADP agonist;
providing a second sample of fresh whole blood of the subject;
measuring a level of hematocrit and/or hemoglobin of the second sample; and
transforming the apparent level of platelet reactivity by the level of hematocrit and/or hemoglobin to assess thrombosis risk of a subject.

8. The method of claim 8, wherein the first sample of fresh whole blood and the second sample of fresh whole blood are obtained from the subject at the same time.

9. The method of claim 8, wherein the first sample of fresh whole blood and the second sample of fresh whole blood are obtained from the subject at different times less than 48 hours part.

10. The method of claim 8, wherein measuring an apparent level of platelet reactivity induced by the adenosine diphosphate agonist generates a PRU value.

11. The method of claim 12, wherein transforming the apparent level of platelet reactivity by the level of hematocrit and/or hemoglobin to obtain the actual platelet reactivity of the subject comprises applying a correction algorithm to normalize a PRU value by the level of hematocrit and/or hemoglobin.

12. The method of claim 13, wherein normalizing a PRU value comprises subtracting a fixed value of PRU for every hematocrit percentage point below and adding the fixed value of PRU for every percentage point above 40%.

13. A method for assessing thrombosis risk of a subject in response to ticagrelor therapy, comprising:
providing a first sample of fresh whole blood of the subject;
applying an adenosine diphosphate agonist to the sample to induce platelet activation;
measuring an apparent level of platelet reactivity induced by the ADP agonist;
providing a second sample of fresh whole blood of the subject;
measuring a level of hematocrit and/or hemoglobin of the second sample; and
transforming the apparent level of platelet reactivity by the level of hematocrit and/or hemoglobin to assess thrombosis risk of a subject.

14. The method of claim 15, wherein the first sample of fresh whole blood and the second sample of fresh whole blood are obtained from the subject at the same time.

15. The method of claim 15, wherein the first sample of fresh whole blood and the second sample of fresh whole blood are obtained from the subject at different times less than 48 hours part.

16. The method of claim 15, wherein measuring an apparent level of platelet reactivity induced by the adenosine diphosphate agonist generates a PRU value.

17. The method of claim 19, wherein transforming the apparent level of platelet reactivity by the level of hematocrit and/or hemoglobin to obtain the actual platelet reactivity of the subject comprises applying a correction algorithm to normalize a PRU value by the level of hematocrit and/or hemoglobin.

18. The method of claim 20, wherein normalizing a PRU value comprises subtracting a fixed value of PRU for every hematocrit percentage point below and adding the fixed value of PRU for every percentage point above 40%.
